(19) United States
(12) Patent Application Publication
(10) Pub. No.: US 2026/0354425 A1
(43) Pub. Date: Nov. 12, 2026

(54) Green Processing Technology For Sea Cucumber Powder With Targeted Enrichment And Enhanced Polysaccharide Release Rate

(71) Applicant: Dalian Polytechnic University, Dalian City (CN)

(72) Inventors: Xiuping DONG, Dalian City (CN); Beiwei ZHU, Dalian City (CN); Zheming WANG, Dalian City (CN); Qiong WU, Dalian City (CN); Jian JIAO, Dalian City (CN); Shuang SONG, Dalian City (CN); Qinggang XU, Dalian City (CN); Xiliang YU, Dalian City (CN)

(21) Appl. No.: 19/719,306

(22) Filed: Jun. 25, 2026

Related U.S. Application Data
(63) Continuation of application No. PCT/CN2025/134668, filed on Nov. 13, 2025.

(30) Foreign Application Priority Data
Nov. 20, 2024 (CN) 2024116626998

(51) Int. Cl. A23L 5/20; A23B 4/03; A23L 17/10; A61K 35/616; B01D 11/02

(52) U.S. Cl. CPC A23L 5/23; A23B 4/03; A23L 17/10; A61K 35/616; B01D 11/0203; B01D 11/0288

(57) ABSTRACT

Disclosed is a green processing technology for sea cucumber powder with targeted enrichment and enhanced polysaccharide release rate, and belongs to the field of food processing. The sea cucumber powder is prepared by the following steps: S1. sea cucumber pretreatment; S2. sea cucumber nutrient enrichment; S3., sea cucumber powder particle size regulation; S4. “single-stage” supercritical treatment; S5. in-situ ultrafine physical pulverization; and S6. “dual-stage” supercritical treatment. By designing a gas disperser and additionally providing a second perforated plate, a supercritical extractor can perform effective supercritical carbon dioxide extraction on the ultrafine sea cucumber powder, thereby achieving deodorization and improving the edible quality. By detecting and regulating a bulk density and a tap density, optimal parameters for processing the ultrafine sea cucumber powder are found, so that the prepared ultrafine sea cucumber powder has a high polysaccharide release rate, good edible quality and easy subsequent processing.

### TECHNICAL FIELD

The present disclosure relates to a green processing technology for a sea cucumber powder capable of achieving directional enrichment and improving a polysaccharide release rate, and belongs to the field of food processing.

### BACKGROUND

Sea cucumber (Stichopus japonicus) is a benthic marine invertebrate belonging to the phylum Echinodermata, class Holothuroidea, and is recognized as a valuable living marine resource. Sea cucumber is known for nutritional characteristics of high protein and low fat thereof, as well as rich trace elements and various amino acids. Sea cucumber polysaccharides are one of important active ingredients in the body wall of sea cucumber, and account for 4-10% of total organic matter in dried sea cucumber. Sea cucumber polysaccharides are acidic viscous polysaccharides with a high biological activity in anticoagulation, blood lipid lowering, immune regulation, anti-aging, and the like. However, sea cucumber polysaccharides are bound to proteins through glycopeptide bonds and hydrogen bonds, so absorption and utilization of the polysaccharides require degradation of the proteins connected to the polysaccharides.

In the prior art, to achieve effective separation of the proteins from the polysaccharides, the concept of ultrafine sea cucumber powder has been introduced. For example, CN 106509694 A, Preparation of a convenient eating and easily digestible ultrafine sea cucumber powder, discloses a ultrafine sea cucumber powder with an improved digestibility and a high retention rate (95.42%) of crude polysaccharides; and CN 105495411 A, A method for preparing sea cucumber ultrafine pure powder tablets, discloses a 1-30 μm sea cucumber ultrafine powder that meets the specification of cell disruption and allows active substances of sea cucumber to be completely released. However, in the two aforementioned patents, the processing technologies of the ultrafine sea cucumber powders are relatively simple, in which sea cucumber is simply washed, sterilized, and enzyme inactivated before being directly processed into ultrafine powders. In the processing technologies, there is still no a deodorization step, a large number of fishy odor molecules still remain in the products, and no specific metrics or standards are provided to characterize or control the particle size of the pulverized sea cucumber powders. Therefore, the final products suffers from poor edible quality, inadequate purity, and uncontrollable product quality.

Other methods for achieving separation of proteins from polysaccharides include protease hydrolysis and extraction with alkaline solution, such as a method disclosed in patent CN 110204626 A “A device and method for separating and refining sea cucumber polysaccharides”. However, the use of the alkaline solution can damage the structures of sea cucumber polysaccharides, causing detachment of glycosyl groups and resulting in changes in the structures. Therefore, the concentration of the alkaline solution and reaction conditions need to be strictly controlled. Enzymatic hydrolysis requires controlling the concentration, temperature, and time of an enzyme reaction, and is complex in operating conditions. Moreover, the products of the enzymatic hydrolysis reaction have undesirable off-flavors that impair sensory quality. Therefore, physical pulverization is superior to enzymatic hydrolysis for this purpose.

Based on the above, further research is needed to advance the extraction and utilization of sea cucumber polysaccharides to produce a sea cucumber polysaccharide product with good edible quality and superior product quality, by using a more excellent and effective method.

### SUMMARY

### Technical Problem

Sea cucumber polysaccharides have high utilization value. To harness it effectively, sea cucumber polysaccharides need to be separated from proteins with physical pulverization and enzymatic hydrolysis being commonly employed methods for this purpose. A physical pulverization method is simpler in steps and easier to control in product quality compared to the enzymatic extraction method. However, physical pulverization methods in the prior art are still simple, and sea cucumber powders produced suffers from poor edible quality, inadequate purity, and uncontrollable product quality. Therefore, the physical pulverization method needs to be improved.

### Technical Content

An objective of the present disclosure is to overcome the shortcomings of the existing technologies for separating sea cucumber polysaccharides from proteins and provide a preparation method for a sea cucumber powder with a high polysaccharide release rate. The present disclosure improves the release rate of sea cucumber polysaccharides by an appropriate physical pulverization technology, and prepares an antiatherogenic sea cucumber powder with a high polysaccharide release rate, and the sea cucumber powder is verified by simulated digestion and animal models.

To achieve the above objective, the present disclosure provides a preparation method for a sea cucumber powder with a high polysaccharide release rate, the sea cucumber powder being prepared by the following steps:
S1. sea cucumber pretreatment: opening sea cucumber in the abdomen, removing the contents from the body cavity of the sea cucumber, and washing the body wall of the sea cucumber with water to obtain pretreated sea cucumber;
S2. sea cucumber nutrient enrichment: placing the pretreated sea cucumber obtained in S1 in warm water for temporary cultivation, and then drying the sea cucumber to obtain dried sea cucumber;
S3. sea cucumber powder particle size regulation: grinding the dried sea cucumber obtained in S2 into 5-30 mesh coarse sea cucumber powder;
S4. single-stage supercritical treatment: placing the coarse sea cucumber powder obtained in S3 into an extraction kettle of a supercritical extractor and performing supercritical carbon dioxide extraction for deodorization;
S5. in-situ ultrafine physical pulverization: the sea cucumber powder deodorized in S4 is subjected to ultrafine pulverization to obtain a powder, where the powder is controlled to have a bulk density of 0.65-0.70 g/mL and a tap density of 0.70-0.80 g/mL; and
S6. “dual-stage” supercritical treatment: placing the sea cucumber powder obtained in S5 into the extraction kettle of the supercritical extractor and performing supercritical carbon dioxide extraction for deodorization, to obtain a sea cucumber powder with a high polysaccharide release rate.

Further, the temperature of the warm water in S2 is 50-60° C.

Further, the temporary cultivation time in S2 is 4-8 h.

Further, in S2, the temporary cultivation in warm water includes: placing the pretreated sea cucumber in warm water at 50-60° C. for 4-8 h, and changing the warm water every 0.5-1 h.

Further, the drying in S2 includes freeze-drying, heat drying, or air drying.

Further, the supercritical extractor in S4 and S6 is a supercritical extractor provided with a gas disperser.

Further, the gas disperser in the supercritical extractor is an improved gas disperser.

Further, the improved gas disperser includes a mounting pipe (1), connecting flanges (2), first perforated plates (3), filter screens (4), support columns (5), baffle plates (6), telescopic rods (7), springs (70), press plates (8), and a second perforated plate (9). the mounting pipe (1) is made of a transparent material to facilitate observation of the internal situation; the outer sides of both the upper and lower parts of the mounting pipe (1) are connected with the connecting flanges (2) to facilitate connection with an external pipeline; the inner sides of both the middle and lower parts of the mounting pipe (1) are connected with the first perforated plates (3); the lower sides of the first perforated plates (3) are connected with the filter screens (4); four support columns (5) are connected between the first perforated plates (3); the upper sides of the support columns (5) are all connected with the baffle plates (6); the baffle plates (6) are all in contact with the mounting pipe (1); the baffle plates (6) are all connected with the telescopic rods (7); the springs (70) are connected between all the fixed ends and telescopic ends of the telescopic rods (7); the telescopic ends of the telescopic rods (7) are all rotatably connected with the press plates (8); the second perforated plate (9) is placed among the baffle plates (6); the press plates (8) are all in contact and match with the second perforated plate (9); the first perforated plate (3) and the second perforated plate (9) have the same shape; and twelve conical members are arranged on the upper side of the second perforated plate (9) to facilitate reflux.

Further, in S4, the parameters of the extraction kettle are set to a temperature of 30-40° C., a pressure of 25-30 MPa, a CO₂ flow rate of 25-30 L/h, and a time of 2-2.5 h.

Further, in S5, precooling is performed before pulverization is performed using a ultrafine pulverizer; and the precooling is performed by using dry ice or liquid nitrogen, or standing in a freezing environment at a temperature of −80 to-20° C.

Further, in S5, the ultrafine pulverization is performed using a 0.3-0.6 mm screen at a rotational speed of 8,000-12,000 rpm.

Further, in S5, a bulk density is 0.67-0.70 g/mL, and a tap density is 0.77-0.80 g/mL.

Specifically optional, in S5, the bulk density is 0.70 g/mL and the tap density is 0.80 g/mL.

Further, in S6, the parameters of the extraction kettle are set to a temperature of 30-40° C., a pressure of 25-30 MPa, a CO₂ flow rate of 25-30 L/h, and a time of 2-2.5 h.

The present disclosure provides a sea cucumber powder with a high polysaccharide release rate prepared by the above method.

The present disclosure provides uses of the sea cucumber powder with a high polysaccharide release rate in the fields of food, health products, or pharmaceutical preparation.

Further, the use in the field of health products is to a prepare health product that helps maintain a healthy level of blood lipids (cholesterol/triglycerides).

Further, the use in the field of pharmaceutical preparation is to prepare a medicament for preventing and treating atherosclerosis diseases.

The present disclosure has the beneficial effects that:

1. At present, a physical pulverization process for separation of sea cucumber polysaccharides from proteins in the prior art is relatively simple and lacks a deodorization step. By designing the gas disperser and additionally providing the second perforated plate, the supercritical extractor can perform effective supercritical carbon dioxide extraction on the ultrafine sea cucumber powder, thereby achieving deodorization of the ultrafine sea cucumber powder and improving the edible quality of the ultrafine sea cucumber powder. Moreover, the design of the second perforated plate can enable reflux of a carbon dioxide fluid for secondary extraction, thereby improving the extraction efficiency, effectively removing fishy molecules from the ultrafine sea cucumber powder, and improving the purity of sea cucumber polysaccharides in a product.

2. At present, a processing technology of a ultrafine sea cucumber powder lacks parameter measurement and quality control. By detecting and regulating a bulk density and a tap density, the present disclosure found optimal parameters for processing the ultrafine sea cucumber powder, so that the prepared ultrafine sea cucumber powder has a high polysaccharide release rate, good edible quality, and easy subsequent processing. In addition, the prepared ultrafine sea cucumber powder also has excellent effects of resisting atherosclerosis and maintaining a healthy level of blood lipids, and is a product with broad market application prospects.

### BRIEF DESCRIPTION OF FIGURES

FIG. 1 is a schematic three-dimensional structural diagram of an improved gas disperser, where 1 denotes a mounting pipe, 2 denotes a connecting flange, 5 denotes a support column, 7 denotes a telescopic rod, 8 denotes a press plate, and 9 denotes a second perforated plate;

FIG. 2 is a schematic three-dimensional partially-sectioned structural diagram of an improved gas disperser, where 1 denotes a mounting pipe, 2 denotes a connecting flange, 3 denotes a first perforated plate, 4 denotes a filter screen, 5 denotes a support column, 6 denotes a baffle plate, 7 denotes a telescopic rod, 8 denotes a press plate, and 9 denotes a second perforated plate;

FIG. 3 is a schematic enlarged three-dimensional structural diagram of the improved gas disperser at part A; where 1 denotes a mounting pipe, 5 denotes a support column, 6 denotes a baffle plate, 7 denotes a telescopic rod, 70 denotes a spring, 8 denotes a press plate, and 9 denotes a second perforated plate;

FIG. 4 shows a supercritical extraction device with a gas disperser mounted, where the arrow indicates the mounting position of the improved gas disperser;

FIG. 5 shows release curves of sea cucumber polysaccharides in Examples 1 and 2 and Comparative Example 1;

FIG. 6 shows cholesterol levels in the blood of high-fat fed mice after intervention with Example 1 and Comparative Example 1 (with different letters indicating significance);

FIG. 7 shows aortic sections of high-fat fed mice after intervention with Example 1 and Comparative Example 1;

FIG. 8 shows the vascular wall thicknesses of high-fat fed mice after intervention with Example 1 and Comparative Example 1 (with different letters indicating significance);

FIG. 9 is a GC-IMS two-dimensional differential topographic map of Example 1;

FIG. 10 is a GC-IMS two-dimensional differential topographic map of Comparative Example 3; and

FIG. 11 is a GC-IMS two-dimensional differential topographic map of Comparative Example 4.

### DETAILED DESCRIPTION

The technical solutions in examples of the present disclosure will be clearly and completely described below with reference to the accompanying drawings in the examples of the present disclosure. Apparently, the described examples are merely a part, rather than all, of the examples of the present disclosure. All other examples obtained by a person of ordinary skill in the art based on the disclosed examples without creative efforts shall fall within the protection scope of the present disclosure.

In the present disclosure, unless otherwise explicitly specified and limited, the terms “link”, “connect”, and “fix” should be understood in a broad sense. For example, a connection may be a fixed connection, a detachable connection, or an integral connection; a mechanical connection, or an electrical connection; a direct connection, or an indirect connection through an intermediate, or an internal communication between two elements, or an interaction between two elements. For those of ordinary skill in the art, the specific meanings of the above terms in the present disclosure can be understood according to specific situations.

In the present disclosure, unless otherwise explicitly specified and defined, a first feature being “above” or “below” a second feature may include the first and second features being in direct contact, or may include the first and second features being not in direct contact but in contact through another feature therebetween. In addition, a first feature being “on”, “above”, or “over” a second feature includes the first feature being right above or obliquely above the second feature, or merely indicates the horizontal height of the first feature being greater than that of the second feature. A first feature being “below”, “under”, and “beneath” a second feature includes the first feature being right below and obliquely below the second feature, or simply indicates the horizontal height of the first feature being less than that of the second feature.

### Experimental Instruments

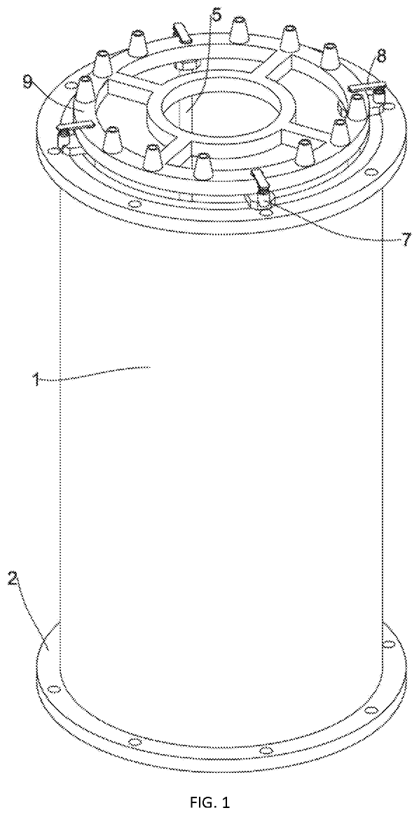

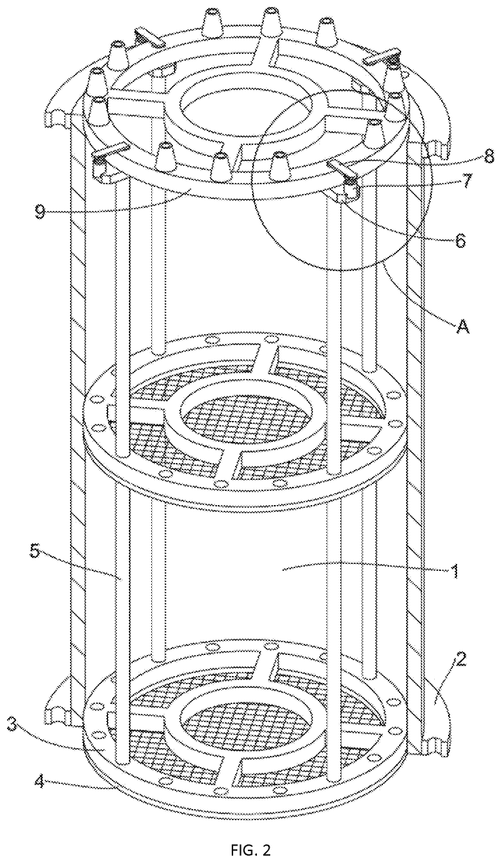

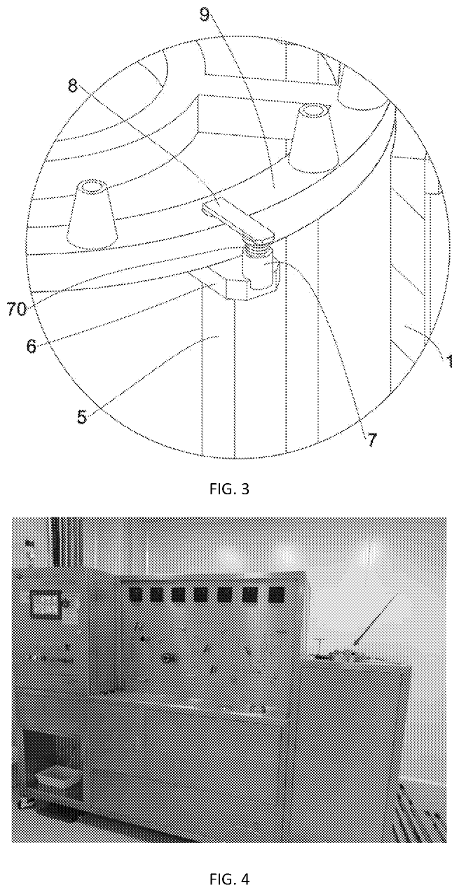

A supercritical extractor with an improved gas disperser used in the examples and comparative examples is manufactured by Jiangsu Gaoke Pharmaceutical Equipment Co., Ltd., with the model number GKSCFE220-40-30L. The gas disperser of the supercritical extractor has been improved, and the improved gas disperser is shown in FIGS. 1-3.

The improved gas disperser includes a mounting pipe 1, connecting flanges 2, first perforated plates 3, filter screens 4, support columns 5, baffle plates 6, telescopic rods 7, springs 70, press plates 8, and a second perforated plate 9.

The mounting pipe 1 is made of a transparent material to facilitate observation of the internal situation. The outer sides of both the upper and lower parts of the mounting pipe 1 are connected with the connecting flanges 2 to facilitate connection with an external pipeline. The inner sides of both the middle and lower parts of the mounting pipe 1 are connected with the first perforated plates 3. The lower sides of both the first perforated plates 3 are connected with the filter screens 4. The four support columns 5 are connected between the first perforated plates 3. The upper sides of the support columns 5 are all connected with the baffle plates 6. The baffle plates 6 are all in contact with the mounting pipe 1. The baffle plates 6 are all connected with the telescopic rods 7. The springs 70 are connected between all the fixed ends and telescopic ends of the telescopic rods 7. The telescopic ends of the telescopic rods 7 are all rotatably connected with the press plates 8. The second perforated plate 9 is placed among the baffle plates 6. The press plates 8 are all in contact and match with the second perforated plate 9. The first perforated plate 3 and the second perforated plate 9 have the same shape. 12 conical members are arranged on the upper side of the second perforated plate 9 to facilitate reflux.

When supercritical carbon dioxide extraction is performed, an improved gas disperser is used, the device is brought to a location where an extracted component needs to be extracted, so that the connecting flanges 2 at the lower part of the mounting pipe 1 are connected to a feed pipeline. The mounting pipe 1 is made of a transparent material to facilitate observation of the internal situation. The press plates 8 are pulled to move and rotate, causing the telescopic rods 7 to extend and the springs 70 to be stretched. Next, the second perforated plate 9 is brought into contact with the baffle plates 6. Then the press plates 8 are rotated in an opposite direction to reset. After releasing the press plates 8, the springs 70 rebound, and the telescopic rods 7 contract and recover, so that the press plates 8 come into contact with the second perforated plate 9, thereby fixing the second perforated plate 9 and preventing the second perforated plate 9 from falling off due to flow of a carbon dioxide fluid. Then, the upper connecting flange 2 is connected to a feed inlet of the extraction tank. By adding the carbon dioxide fluid into the feed pipeline, the carbon dioxide fluid enters the mounting pipe 1, flows through the first perforated plate 3, the filter screens 4, and the second perforated plate 9, and then flows into the extraction tank for material extraction. The first perforated plate 3 and the second perforated plate 9 have the same shape, thereby enabling uniform feeding of the carbon dioxide fluid, improving the efficiency of extraction and production, and allowing the device to be placed inside the extraction tank. The carbon dioxide can achieve the effect of reflux under the action of the conical members, thereby causing the carbon dioxide fluid to reflux under the action of the conical members on the second perforated plate 9 to perform secondary extraction, facilitating the secondary extraction of a material, lowering the pressure of the carbon dioxide fluid when the carbon dioxide fluid is discharged from a material basket, preventing the material from being carried away, and improving the extraction effect.

### Example 1

S1. Sea cucumber pretreatment: Fresh sea cucumber was opened in the abdomen, the contents were removed from the body cavity of the sea cucumber, and the body wall of the sea cucumber was washed with water.

S2. Sea cucumber nutrient enrichment: The pretreated sea cucumber obtained in S1 was placed in warm water at 60° C. for temporary cultivation for 6 h, the water was changed every 0.5 h to remove salt from the sea cucumber, and then the sea cucumber was taken out and dried by a vacuum freeze drier for 24 h to obtain dried sea cucumber.

S3. Sea cucumber powder particle size regulation: The dried sea cucumber was pulverized into 20 mesh coarse sea cucumber powder in a pulverizer, ball mill, or crusher.

S4. “Single-stage” supercritical treatment: The coarse sea cucumber powder was deodorized using the supercritical extractor provided with the improved gas disperser. The coarse sea cucumber powder obtained in S3 was placed in an extraction kettle of the supercritical extractor and extracted at a temperature of 30° C., a pressure of 30 MPa, and a CO₂ flow rate of 30 L/h for 2.5 h. Following the extraction, the sea cucumber powder was collected from the extraction kettle and stored for future use.

S5. In-situ ultrafine physical pulverization: The sea cucumber powder obtained in S4 was precooled in liquid nitrogen for 1 h, and then was pulverized using a ultrafine pulverizer at a rotational speed of 12,000 rpm using a 0.6 mm screen. The powder bulk density was controlled to 0.65 g/mL, and the tap density was controlled to 0.73 g/mL.

S6. “Dual-stage” supercritical treatment: The coarse sea cucumber powder was deodorized using the supercritical extractor provided with the improved gas disperser. The sea cucumber powder obtained in S5 was placed in an extraction kettle of the supercritical extractor and extracted at a temperature of 30° C., a pressure of 30 MPa, and a CO₂ flow rate of 30 L/h for 2.5 h. Following the extraction, the sea cucumber powder was collected from the extraction kettle and stored for future use.

S7. The sea cucumber powder obtained in S6 was packaged, sterilized, sealed, and cooled to room temperature to obtain an ultrafine sea cucumber powder product.

### Example 2

S1. Sea cucumber pretreatment: Caught sea cucumber was opened in the abdomen, the contents were removed from the body cavity of the sea cucumber, and the body wall of the sea cucumber was washed with water.

S2. Sea cucumber nutrient enrichment: The pretreated sea cucumber obtained in S1 was placed in warm water at 60° C. for temporary cultivation for 6 h, the water was changed every 0.5 h to remove salt from the sea cucumber, and then the sea cucumber was taken out and dried by a vacuum freeze drier for 24 h to obtain dried sea cucumber.

S3. Sea cucumber powder particle size regulation: The dried sea cucumber was pulverized into 20 mesh coarse sea cucumber powder in a pulverizer, ball mill, or crusher.

S4. “Single-stage” supercritical treatment: The coarse sea cucumber powder was deodorized using the supercritical extractor provided with the improved gas disperser. The coarse sea cucumber powder obtained in S3 was placed in an extraction kettle of the supercritical extractor and extracted at a temperature of 30° C., a pressure of 30 MPa, and a CO₂ flow rate of 30 L/h for 2.5 h. Following the extraction, the sea cucumber powder was collected from the extraction kettle and stored for future use.

S5. In-situ ultrafine physical pulverization: The sea cucumber powder obtained in S4 was precooled in liquid nitrogen for 1 h, and then was pulverized using a ultrafine pulverizer at a rotational speed of 12,000 rpm using a 0.6 mm screen. The powder bulk density was controlled to 0.70 g/mL, and the tap density was controlled to 0.80 g/mL.

S6. “Dual-stage” supercritical treatment: The coarse sea cucumber powder was deodorized using the supercritical extractor provided with the improved gas disperser. The sea cucumber powder obtained in S5 was placed in an extraction kettle of the supercritical extractor and extracted at a temperature of 30° C., a pressure of 30 MPa, and a CO₂ flow rate of 30 L/h for 2.5 h. Following the extraction, the sea cucumber powder was collected from the extraction kettle and stored for future use.

S7. The sea cucumber powder obtained in S6 was packaged, sterilized, sealed, and cooled to room temperature to obtain an ultrafine sea cucumber powder product.

### Example 3

S1. Sea cucumber pretreatment: Caught sea cucumber was opened in the abdomen, the contents were removed from the body cavity of the sea cucumber, and the body wall of the sea cucumber was washed with water.

S2. Sea cucumber nutrient enrichment: The pretreated sea cucumber obtained in S1 was placed in warm water at 60° C. for temporary cultivation for 6 h, the water was changed every 0.5 h to remove salt from the sea cucumber, and then the sea cucumber was taken out and dried by a vacuum freeze drier for 24 h to obtain dried sea cucumber.

S3. Sea cucumber powder particle size regulation: The dried sea cucumber was pulverized into 20 mesh coarse sea cucumber powder in a pulverizer, ball mill, or crusher.

S4. “Single-stage” supercritical treatment: The coarse sea cucumber powder was deodorized using the supercritical extractor provided with the improved gas disperser. The coarse sea cucumber powder obtained in S3 was placed in an extraction kettle of the supercritical extractor and extracted at a temperature of 30° C., a pressure of 30 MPa, and a CO₂ flow rate of 30 L/h for 2.5 h. Following the extraction, the sea cucumber powder was collected from the extraction kettle and stored for future use.

S5. In-situ ultrafine physical pulverization: The sea cucumber powder obtained in S4 was precooled in liquid nitrogen for 1 h, and then was pulverized using a ultrafine pulverizer at a rotational speed of 12,000 rpm using a 0.6 mm screen. The powder bulk density was controlled to 0.75 g/mL, and the tap density was controlled to 0.87 g/mL.

S6. “Dual-stage” supercritical treatment: The coarse sea cucumber powder was deodorized using the supercritical extractor provided with the improved gas disperser. The sea cucumber powder obtained in S5 was placed in an extraction kettle of the supercritical extractor and extracted at a temperature of 30° C., a pressure of 30 MPa, and a CO₂ flow rate of 30 L/h for 2.5 h. Following the extraction, the sea cucumber powder was collected from the extraction kettle and stored for future use.

S7. The sea cucumber powder obtained in S6 was packaged, sterilized, sealed, and cooled to room temperature to obtain an ultrafine sea cucumber powder product.

### Example 4

S1. 50 g of the ultrafine sea cucumber powder prepared in Example 1 and 30 g of microcrystalline cellulose were weighed, put into a mixer, and mixed at a rotational speed of 1,000 r/min for 15 min to evenly disperse the two powders.

S2. 2 g of magnesium stearate was added to the mixer, the rotational speed was adjusted to 800 r/min, and mixing was continued for 5 min to ensure that the magnesium stearate evenly covered the surface of the powder and reduce wall adhesion during a filling process.

S3. Empty capsules were balanced in an environment of a temperature of 25° C. and a relative humidity of 45-55% for 2 h. Then a capsule filling machine was started, a filling amount was adjusted to 0.5 g/capsule (in which the content of the ultrafine sea cucumber powder was about 0.3 g/capsule), and the capsule filling machine was set to a rotational speed of 30 r/min and a pressure of 0.3 MPa, to obtain capsules for helping maintain a healthy level of blood lipids or prevent and treat atherosclerosis.

### Comparative Example 1

S1. Sea cucumber pretreatment: Caught sea cucumber was opened in the abdomen, the contents were removed from the body cavity of the sea cucumber, and the body wall of the sea cucumber was washed with water.

S2. Sea cucumber nutrient enrichment: The pretreated sea cucumber obtained in S1 was placed in warm water at 60° C. for temporary cultivation for 6 h, the water was changed every 0.5 h to remove salt from the sea cucumber, and then the sea cucumber was taken out and dried by a vacuum freeze drier for 24 h to obtain dried sea cucumber.

S3. Sea cucumber powder particle size regulation: The dried sea cucumber was pulverized into 20 mesh coarse sea cucumber powder in a pulverizer.

### Comparative Example 2

S1. Sea cucumber pretreatment: Fresh sea cucumber was opened in the abdomen, the contents were removed from the body cavity of the sea cucumber, and the body wall of the sea cucumber was washed with water. Then the sea cucumber was taken out and dried by a vacuum freeze drier for 24 h to obtain dried sea cucumber.

S2. Sea cucumber powder particle size regulation: The dried sea cucumber was pulverized into 20 mesh coarse sea cucumber powder in a pulverizer, ball mill, or crusher.

Referring to an experimental method of Zhang Tongtong (Zhang Tongtong. Optimization of Sea Cucumber Polysaccharide Determination and Its In Vivo Metabolism Study [D]. Dalian Polytechnic University, 2023), 10 μL of a sample solution was added to an enzyme-linked immunosorbent assay (ELISA) plate, and then 200 μL of a DMB staining solution (a 42.8 mg/L DMB solution was accurately prepared and adjusted to a pH of 3.3 with formic acid to obtain a solution A, and the solution A was mixed with a 2 mol/L Tris solution at a ratio of 10:1 to obtain the DMB staining solution) was added, shaken, placed in the dark at room temperature for 15 min, and then measured for the absorbance at 525 nm. The content of sulfated polysaccharides in a sample was calculated using a standard curve (y=1.625x+0.4439, R²=0.9989).

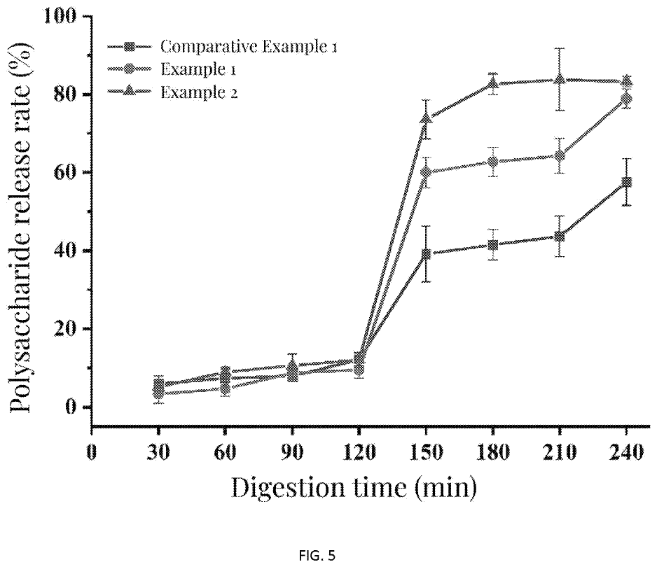

The sea cucumber powders from Example 1, Example 2, and Comparative Example 1 were tested as described above. The results of the release rate of sea cucumber polysaccharides are shown in FIG. 5, from which it can be seen that the release rate of sea cucumber polysaccharides was significantly improved after ultrafine pulverization. The sea cucumber polysaccharides mainly bound to proteins, making it difficult for them to be released into digestive juices and subsequently utilized by the human body. Nevertheless, the physical pulverization technology provided by the examples can increase the contact area with digestive enzymes by changing the specific surface area of the sea cucumber polysaccharides, to provide more enzyme cleavage sites and improve the release rate of the sea cucumber polysaccharides. Compared to Comparative Example 1, the polysaccharide release rate of Example 1 increased by 21%.

As the bulk density and the tap density gradually increase, the particle size of the ultrafine sea cucumber powder gradually decreases. The particle size of the ultrafine sea cucumber powder in Example 3 has reached a very small degree, which also means that processing to such a degree is already difficult, and the polysaccharide release rate cannot be further improved. The polysaccharide release rate of the ultrafine sea cucumber powder prepared in Example 3, as measured by the above experiment, is not significantly different from the result of Example 2. In addition, a microstructure experiment has shown that as the particle size decreased and the electrostatic force increased, the ultrafine powder was more likely to aggregate, which can affect subsequent product processing and also affect the edible quality of a product. Therefore, controlling the bulk density and the tap density to around 0.70 g/mL and 0.80 g/mL yield the best results and they should not be further increased. At this time, the product has a relatively high polysaccharide release rate, a low degree of processing difficulty, and an optimal edible quality.

### Contents of Animal Experiment

### 1. Experimental Animals:

36 APOE mice, male, 18-22 g; and 6 C57 mice, male, 18-22 g; from Liaoning Changsheng Biotechnology Co., Ltd., with an experimental animal production license number: NO.SCXK (LIAO) 2020-0001.

### 2. Experimental Grouping and Treatment

### Experimental Grouping and Treatment

(1) Normal control group: 8 C57 mice, fed normally without gavage.

The background strain of the APOE mice is C57 mice, and APOE is obtained by knocking out the APOE gene under the background of the C57 mice. Generally, C57 mice are used as blank controls.

(2) Model group: 8 APOE mice, fed with a high-fat diet without gavage.

(3) Positive control group: 8 APOE mice, fed with a high-fat diet, and administered atorvastatin via oral gavage (1.8 mg/kg), once a day for 8 weeks at a dose of 300 uL.

(4) High-dose group of Comparative Example 1:8 APOE mice, fed with a high-fat diet, and administered with a high dose of common sea cucumber powder administered atorvastatin via oral gavage (2 mg/g), once a day for 8 weeks.

(5) Low-dose group of Comparative Example 1:8 APOE mice, fed with a high-fat diet, and administered with a low dose of common sea cucumber powder administered atorvastatin via oral gavage (1 mg/g), once a day for 8 weeks.

(6) High-dose group of Example 1:8 APOE mice, fed with a high-fat diet, and administered with a high dose of ultrafine sea cucumber powder administered atorvastatin via oral gavage (2 mg/g), once a day for 8 weeks.

(7) Low-dose group of Example 1:8 APOE mice, fed with a high-fat diet, and administered with a low dose of ultrafine sea cucumber powder administered atorvastatin via oral gavage (1 mg/g), once a day for 8 weeks.

Collection of samples after 8 weeks of gavage: Sera (4 blood lipid tests), was collected along with hearts, livers, and aortas.

Referring to an experimental method of Cao Xu (Cao Xu. Study on extraction process and anti-atherosclerosis effect and mechanism of total triterpene acids from Ganoderma lucidum spores [D]. Xihua University, 2021.), blood samples were placed at room temperature for 2 h and then centrifuged at 4° C. and 3,000 rpm for 15 min, and the supernatant was immediately taken and detected.

Referring to an experimental method of Tian Guangjing (Tian Guangjing. Study on improvement effect and mechanism of flax oil on atherosclerosis [D]. Chinese Academy of Agricultural Sciences, 2018), aortic tissue was dissected from mice and fixed in 4% paraformaldehyde. The aortas and livers were stained with hematoxylin and eosin (H&E) to evaluate pathological lesions.

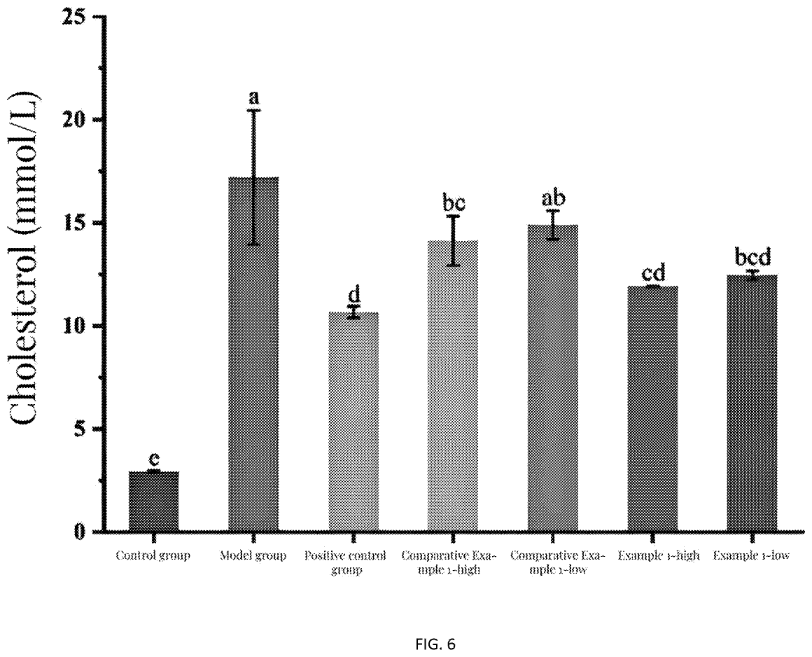

A high-fat diet may lead to atherosclerosis and result in specific changes in cholesterol. Example 1 and Comparative Example 1 were evaluated for intervention effects on high-fat induced atherosclerosis in mice, and the serum lipid levels of mice in each group were measured, as shown in FIG. 6. From FIG. 6, it can be seen that the ultrafine sea cucumber powder of Example 1 can significantly reduce the blood lipid levels of mice. This also confirms that the physical pulverization technology provided by the present disclosure cannot change the biological activity of the sea cucumber polysaccharides.

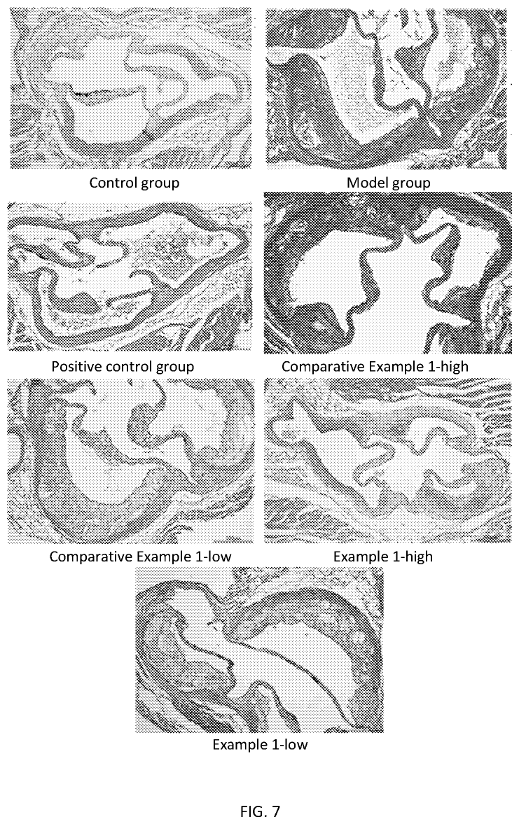

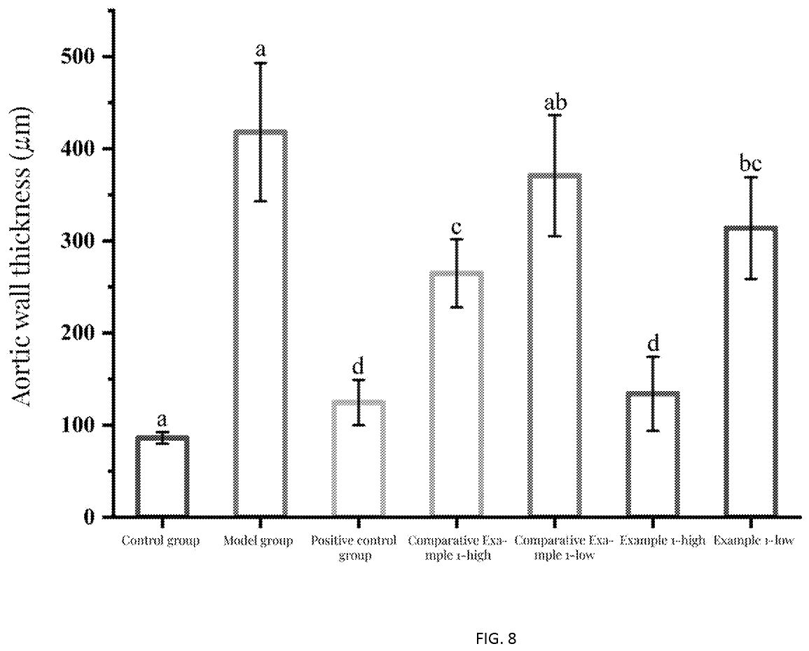

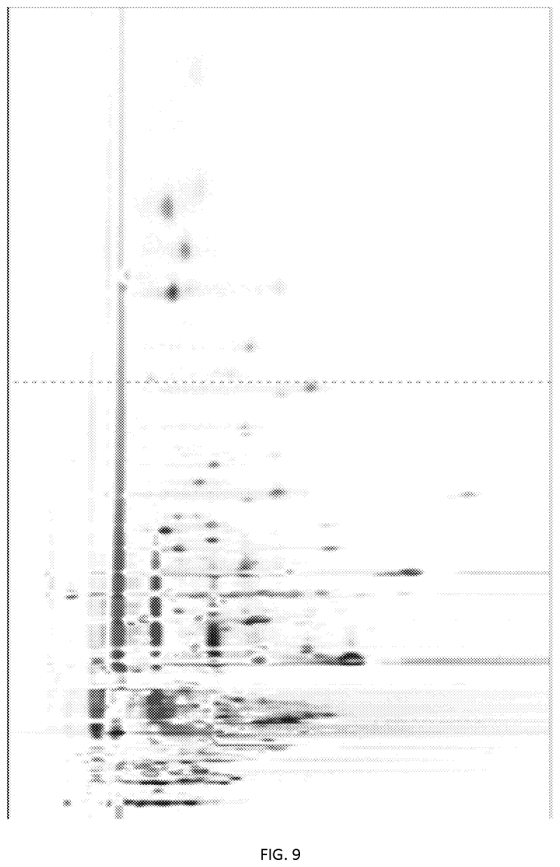

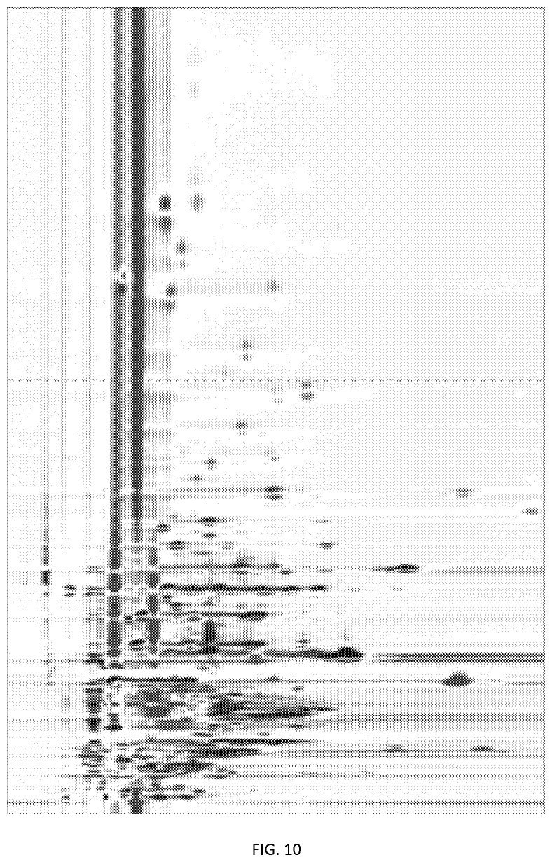

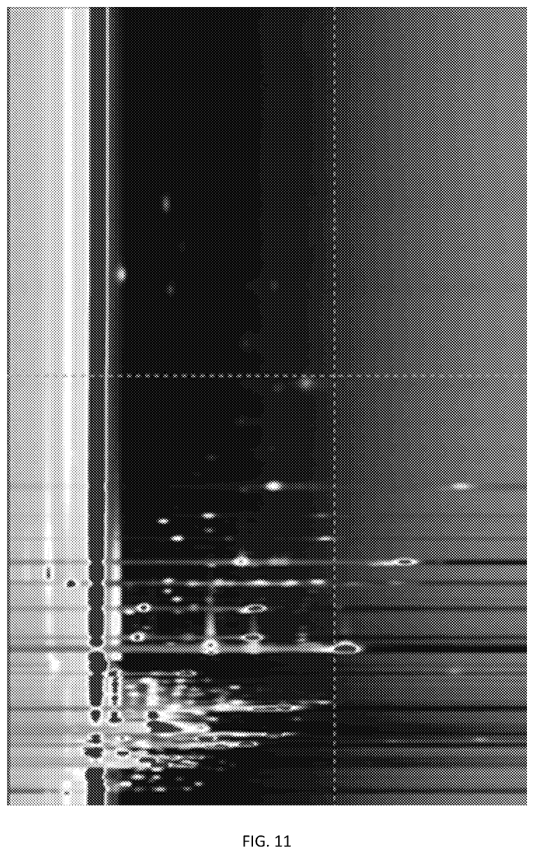

Histological changes of aortic vessels are the key changes of atherosclerosis, which are mainly manifested as thickening of arterial walls and accumulation of vascular lipids. Aortic sections (FIG. 7) and wall thickness (FIG. 8) from Example 1 and Comparative Example 1 are presented. It can be seen that the mice fed with the high-fat diet had serious aortic tissue lesions, with formation of significant atherosclerotic plaques, massive formation and accumulation of foam cells that protrude into the lumen, thickening of aortic intima, and obvious spaces in endothelial cells and tunica media. The control group had a significantly increased aortic thickness of 418 μm. Compared with Comparative Example 1, Example 1 has a better interventive effect on atherosclerosis.

### Comparative Example 3

Referring to the preparation method in Example 1, an ultrafine sea cucumber powder product was prepared by performing S1, S2, S3, and S4 only.

### Comparative Example 4

Referring to the preparation method in Example 1, an ultrafine sea cucumber powder product was prepared by performing S1, S2, S3, S4, and S5 only.

### Comparative Example 5

Referring to the preparation method in Example 1, the supercritical extractor provided with the improved gas disperser was replaced with a conventional supercritical extractor (without improving the gas disperser and lacking design of the second perforated plate). During the extraction process in S6, a pipeline was blocked, and the extraction could not be completed normally. This is because the ultrafine sea cucumber powder is too small in volume, and is easily carried out of a material basket by the carbon dioxide fluid and enters a carbon dioxide pipeline, causing blockage of the carbon dioxide pipeline. The unextracted sea cucumber powder still has a strong fishy odor, with many residual odoriferous impurities, and the purity of a product is low.

Referring to an experimental method of Xu Jialin (Xu Jialin, Peng Jian, Xu Yujuan, et al. Study on physicochemical and powder characteristics of ultrafine mango powder with different addition amounts of peel [J]. Food and Fermentation Industries, 2024, 1-13.), a 2 mL EP tube was taken and filled with the sea cucumber powder. The mass M of the sea cucumber powder in the container was recorded, and the bulk density was calculated by the following formula: bulk density (g/mL)=M/2. The higher the bulk density value, the smaller the gaps between particles, and the smaller the particle size.

Referring to the experimental method of Li Zhenjiang (Li Zhenjiang, Liu Ying, Zhi Li, et al. Effect of ball milling ultrafine pulverization on particle characteristics and polysaccharide extraction efficiency of Inonotus obliquus powder [J]. China Food Additives, 2021, 32 (11): 1-8.), a 2 mL EP tube was taken and filled with the sea cucumber powder. The EP tube was continuously tapped and the sample was continuously added until the powder weight reached a stable state. The mass M₁ of the sea cucumber powder in the container was recorded, and the tap density was calculated by the following formula: tap density (g/mL)=M₁/2. The higher the tap density, the smaller the particle gap, the smaller the total volume, and the smaller the particle size.

Referring to an experimental method of Qi Baokun (Qi Baokun, Liu Yuwen, Yao Yuxie, et al. Analysis of effect of protease on volatile flavor of soybean meal based on HS-GC-IMS and HS-SPME-GC-MS [J]. Journal of Agricultural Machinery, 2024, 1-27.), 0.1 g of the sea cucumber powder was added to 1 mL of deionized water, followed by placement in a 20 mL headspace sampling bottle. After incubation at 60° C. for 30 min, automatic injection was performed with an autosampler syringe at 85° C. and an injection volume of 500 μL in a splitless mode.

The chromatographic conditions include: a WAX capillary column, a column temperature of 60° C., an operating time of 30 min, a purity of a carrier gas N₂≥99.999%, and a purity of N₂≥99.999%. The analysis conditions were: 2 mL/min for 2 min, 10 mL/min for 8 min, 100 mL/min for 10 min, and 150 mL/min for 10 min (end).

Qualitative analysis of different volatile substances: A drift gas (nitrogen) was set at 150 mL/min. Each spectrum was scanned 12 times on average. All analyses were conducted in triplicate. The retention index (RI) of VC was calculated using N-ketone C4-C9 (Beijing Sinopharm Chemical Reagent Co., Ltd) as an external reference. Qualitative analysis was performed by comparing the drift times of standard samples in RI and GC-IMS libraries.

The ultrafine sea cucumber powder prepared in Example 1 and the sea cucumber powders prepared in Comparative Examples 3 and 4 were tested referring to the above methods for testing processes. The GC-IMS two-dimensional differential topographic map is shown in FIGS. 5-7. The darker the color, the higher the concentration of characteristic flavors in a sample. From the figure, it can be seen that the green deodorization technology provided by the present disclosure effectively removed fishy odor substances from a sample, and the difference in the overall flavor of the sample after deodorization was not significant. From the results of Comparative Examples 3 and 4, it can be seen that the ultrafine pulverization process increased the concentration of volatile substances in the sample, so further deodorization was required after ultrafine pulverization, while a conventional supercritical extractor could not achieve deodorization of the ultrafine sea cucumber powder.

The examples provided above are not intended to limit the scope covered by the present disclosure, nor are the steps described to limit the execution order. Apparent improvements made to the present disclosure by those skilled in the art based on existing common knowledge also fall within the scope of protection defined in the claims of the present disclosure.

What is claimed is:

1. A preparation method for a sea cucumber powder with a high polysaccharide release rate, the sea cucumber powder being prepared by the following steps:
S1. sea cucumber pretreatment: opening sea cucumber in the abdomen, removing contents from the body cavity of the sea cucumber, and washing the body wall of the sea cucumber with water to obtain pretreated sea cucumber;
S2. sea cucumber nutrient enrichment: placing the pretreated sea cucumber obtained in S1 in warm water for temporary cultivation, and then drying the sea cucumber to obtain dried sea cucumber;
S3. sea cucumber powder particle size regulation: grinding the dried sea cucumber obtained in S2 into 5-30 mesh coarse sea cucumber powder;
S4. single-stage supercritical treatment: placing the coarse sea cucumber powder obtained in S3 into an extraction kettle of a supercritical extractor and performing supercritical carbon dioxide extraction for deodorization;
S5. in-situ ultrafine physical pulverization: subjecting the sea cucumber powder deodorized in S4 to ultrafine pulverization to obtain a powder, where the powder is controlled to have a bulk density of 0.65-0.70 g/mL and a tap density of 0.70-0.80 g/mL; and
S6. “dual-stage” supercritical treatment: placing the sea cucumber powder obtained in S5 into the extraction kettle of the supercritical extractor and performing supercritical carbon dioxide extraction for deodorization, to obtain a sea cucumber powder with a high polysaccharide release rate; supercritical carbon dioxide extraction refers to extraction using carbon dioxide fluid whose temperature and pressure above its critical values as the solvent;
wherein in S4 and S6, the parameters of the extraction kettle are set to a temperature of 30-40° C., a pressure of 25-30 MPa, a CO₂ flow rate of 25-30 L/h, and a time of 2-2.5 hours;
in S4 and S6, the supercritical extractor is a supercritical extractor provided with a gas disperser; the gas disperser is an improved gas disperser; the improved gas disperser is placed inside the extraction tank;
the improved gas disperser comprises a mounting pipe (1), connecting flanges (2), first perforated plates (3), filter screens (4), support columns (5), baffle plates (6), telescopic rods (7) springs (70), press plates (8), and a second perforated plate (9);
the mounting pipe (1) is made of a transparent material to facilitate observation of the internal situation; the outer sides of both the upper and lower parts of the mounting pipe (1) are connected with the connecting flanges (2) to facilitate connection with an external pipeline; the inner sides of both the middle and lower parts of the mounting pipe (1) are connected with the first perforated plates (3); the lower sides of the first perforated plates (3) are connected with the filter screens (4); four support columns (5) are connected between the first perforated plates (3); the upper sides of the support columns (5) are all connected with the baffle plates (6); the baffle plates (6) are all in contact with the mounting pipe (1); the baffle plates (6) are all connected with the telescopic rods (7); the springs (70) are connected between all the fixed ends and telescopic ends of the telescopic rods (7); the telescopic ends of the telescopic rods (7) are all rotatably connected with the press plates (8); the second perforated plate (9) is placed among the baffle plates (6); the press plates (8) are all in contact and match with the second perforated plate (9); the first perforated plate (3) and the second perforated plate (9) have the same shape; and twelve conical members are arranged on the upper side of the second perforated plate (9) to facilitate reflux.

2. The preparation method according to claim 1, wherein in S2, the temporary cultivation in warm water includes: placing the pretreated sea cucumber in warm water at 50-60° C. for 4-8 hours, and changing the warm water every 0.5-1 hour.

3. The preparation method according to claim 1, wherein the drying in S2 includes freeze-drying, heat drying, or air drying.

4. The preparation method according to claim 1, wherein in S5, precooling is performed before pulverization is performed using an ultrafine pulverizer; and the precooling is performed by using dry ice or liquid nitrogen, or standing in a freezing environment.

5. The preparation method according to claim 1, wherein in S5, the ultrafine pulverization is performed using a 0.3-0.6 mm screen at a rotational speed of 8,000-12,000 rpm.

6. The preparation method according to claim 1, wherein in S5, a bulk density is 0.67-0.70 g/mL, and a tap density is 0.77-0.80 g/mL.

7. A sea cucumber powder with a high polysaccharide release rate, being prepared by the preparation method according to claim 1.
